(19) United States
(12) Patent Application Publication
(10) Pub. No.: US 2026/0078868 A1
(43) Pub. Date: Mar. 19, 2026

(54) SCENTED HOOK ASSEMBLY

(71) Applicant: Blaine Wagner, Petersburg, MI (US)

(72) Inventor: Blaine Wagner, Petersburg, MI (US)

(21) Appl. No.: 19/302,650

(22) Filed: Aug. 18, 2025

Related U.S. Application Data
(60) Provisional application No. 63/695,398, filed on Sep. 17, 2024.

(51) Int. Cl. F16M 13/02; A61L 9/12

(52) U.S. Cl. CPC F16M 13/022; A61L 9/12; A61L 2209/15

(57) ABSTRACT

A scented hook assembly includes a hook clip, and a scent block removably attached thereto.

### BACKGROUND OF THE INVENTION

This invention relates in general to hooks for supporting articles. In particular, this invention relates to an improved structure for such a hook that can be supported on a support structure and that emits a fragrance or scent.

Hooks are often provided in public restrooms, such as hooks permanently mounted on the wall of a toilet stall. Such hooks allow an occupant of the stall to hang items thereon. Additionally, some public restroom have air fresheners that mask unpleasant smells. Such known air fresheners includes automatic wall mounted aerosol spray dispenses, gel-based air fresheners, and ultrasonic diffusers.

It would however, be desirable to provide an improved structure for such a hook that is strong, easy to install and move, and emits a desired fragrance or scent.

### SUMMARY OF THE INVENTION

This invention relates to an improved structure for a scented hook. In one embodiment, a scented hook includes a body having . . . .

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a scented hook assembly in accordance with this invention.

FIG. 2 is a front elevational view of the scented hook assembly illustrated in FIG. 1.

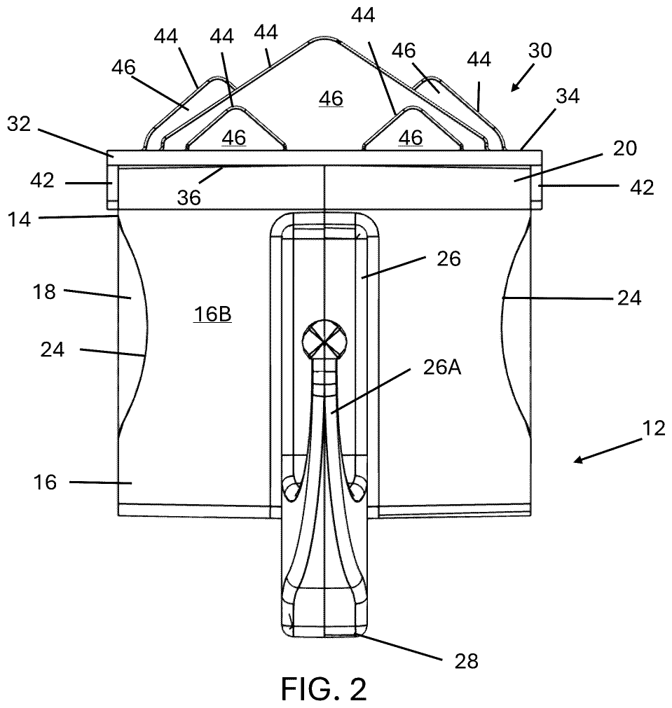

FIG. 3 is a side elevational view of the scented hook assembly illustrated in FIGS. 1 and 2.

FIG. 4 is an alternate perspective view of the scented hook assembly illustrated in FIGS. 1 through 3.

FIG. 5 is a first perspective view of the scent block illustrated FIGS. 1 through 4.

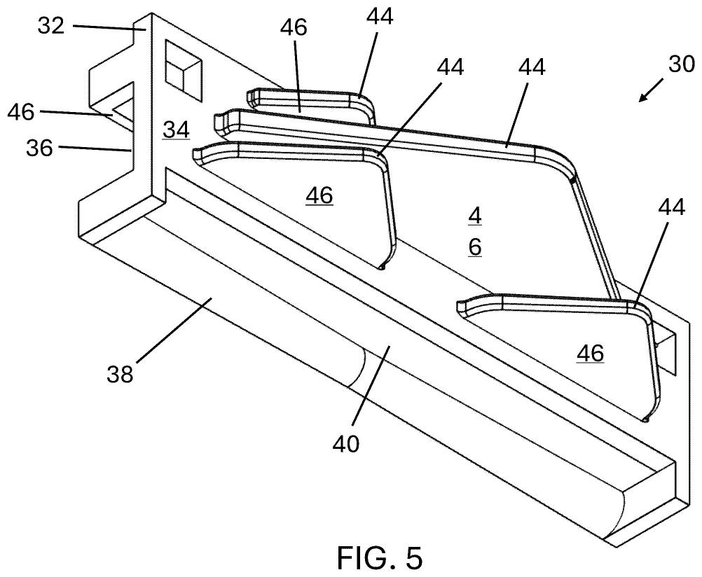

FIG. 6 is a second perspective view of the scent block illustrated FIG. 5.

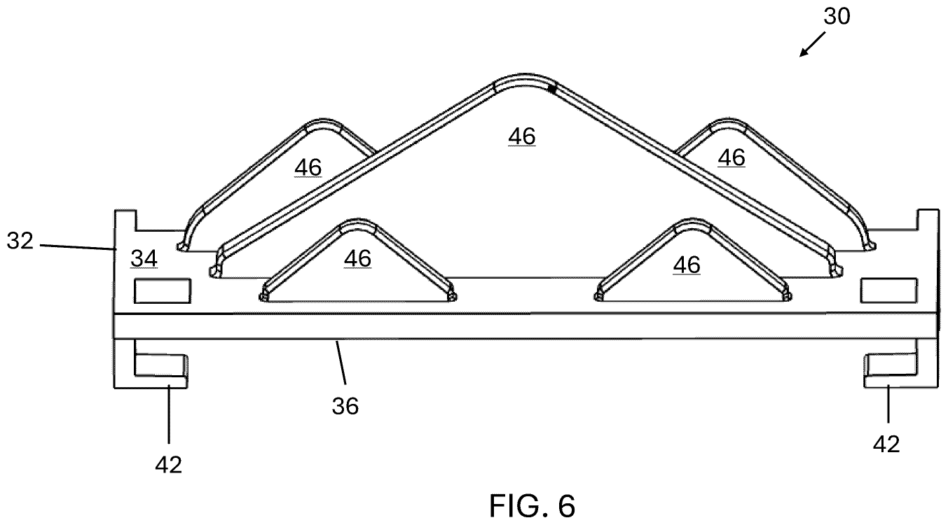

FIG. 7 is a third perspective view of the scent block illustrated FIGS. 4 and 5.

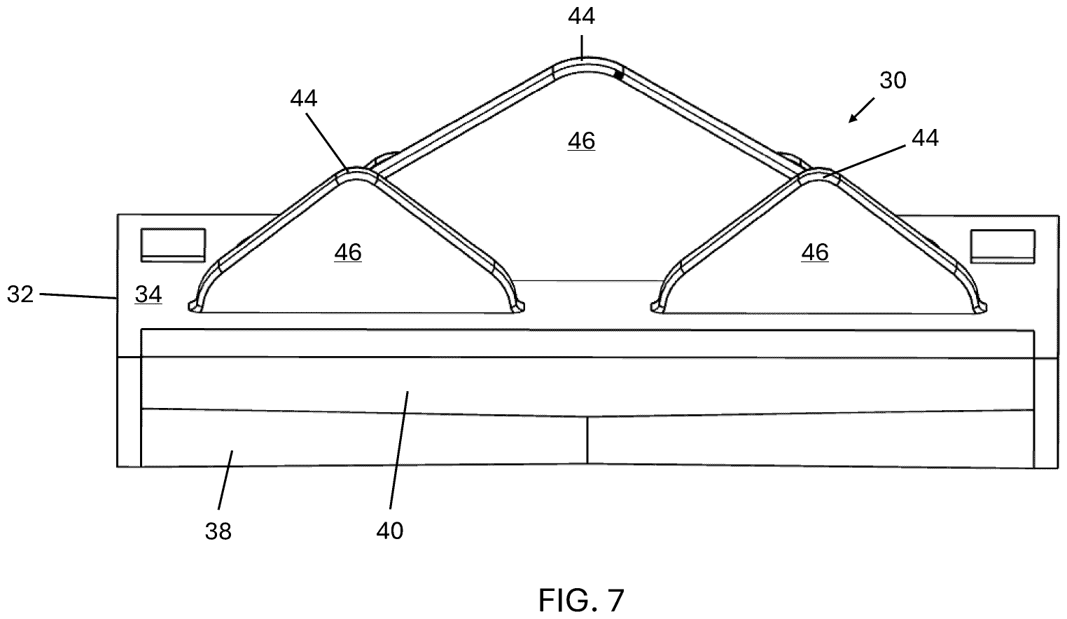

FIG. 8 is a perspective view of the hook clip illustrated in FIGS. 1 through 4.

### DETAILED DESCRIPTION OF THE PREFERRED EMBODIMENT

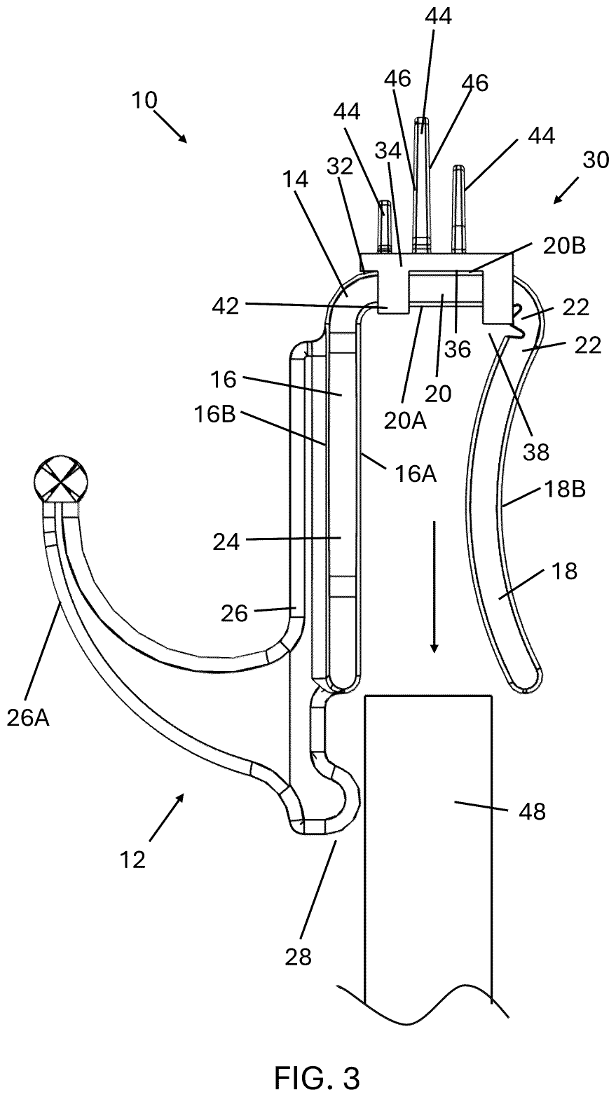

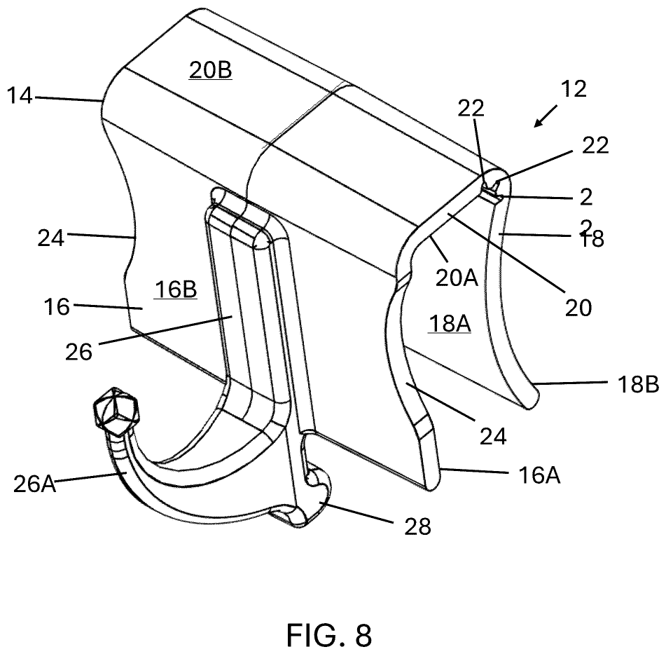

Referring now to the drawings, a scented hook assembly in accordance with this invention is illustrated at 10. The illustrated scented hook assembly 10 includes a hook clip 12 and a scent block 30 removably attached thereto. As best shown in FIGS. 3 and 8, the hook clip 12 includes a body defining a mounting clip 14. The mounting clip 14 includes a first or planar support wall 16 having an inside surface 16A and an outside surface 16B, a second or arcuate wall 18 having an inside surface 18A and an outside surface 18B, and a third wall defining a closed end wall 20 having an inside surface 20A and an outside surface 20B. One or more elongated flex channels 22 are formed at the intersection of the arcuate wall 18 and the closed end wall 20 in one or both of the inside surfaces 18A and 20A thereof. The flex channels 22 allow the arcuate wall 18 to move outwardly when the hook clip 12 is installed on a support structure such as a stall wall panel 48, thus allowing for ease of installation without adversely affecting the hanging strength of the scented hook assembly 10. The flex channels 22 are formed on the inside surfaces 18A and/or 20A and therefore do not catch or collect debris and/or fluids.

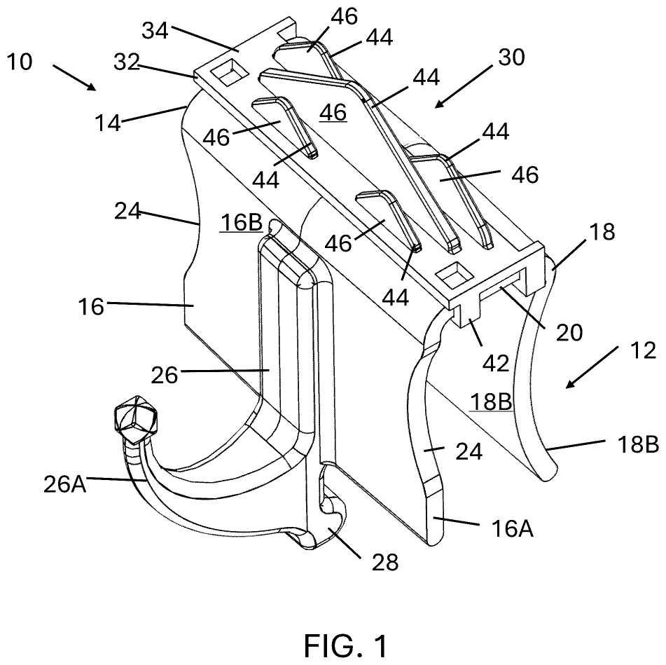

Arcuate indentations are formed on opposing sides of the planar support wall 16 and define finger grips 24. The finger grips 24 assist an installer, such as a restroom maintenance worker, with placement and removal of the scented hook assembly 10 when wearing protective-style gloves. A hook 26 is formed on the outside surface 16B of the planar support wall 16 and includes a outwardly extending hook portion 26A and a hook base extension 28 that extends outwardly from the open end of the planar support wall 16 (downwardly when viewing FIGS. 1 and 3. The hook base extension 28 adds structural strength to the hook 26 when a downward force is applied thereto. The hook base extension 28 also reduces the inflection pressure on the mounting clip 14 to prevent failure or compromise of the mounting clip 14.

The scent block 30 includes a planar base 32 having an outwardly facing first surface 34 and an inwardly facing second surface 36. An elongated bar 38 extends longitudinally parallel to the second surface 36 and defines a mounting slot 40. Two outwardly extending mounting clips 42 are formed on opposing longitudinal ends of the base 32. The first surface 34 of the planar base 32 includes a plurality of outwardly extending fins 44, each fin 44 having opposing flat faces 46. In the illustrated embodiment, five of the fins 44 are shown, each having a triangular shape. However, it will be understood that the scent block 30 may have any desired number of fins 44, and that the fins 44 may have other geometric shapes.

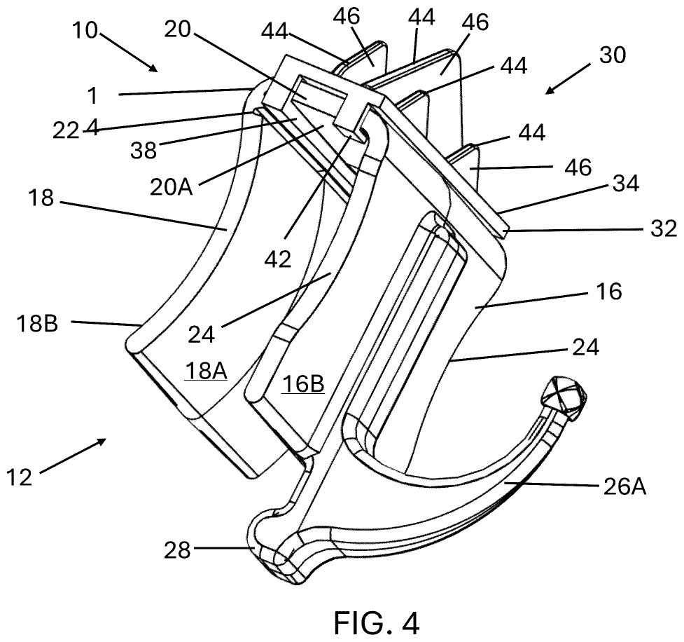

As best shown in FIGS. 1, 3, and 4, the scent block 30 is mounted to the hook clip by extending the arcuate wall 18 through the mounting slot 40 and pushing the mounting clips 42 onto the closed end wall 20 in a snap-fit arrangement, thus defining the scented hook assembly 10.

The scent block 30 may be formed from a strong, lightweight, and durable plastic, including, but not limited to ultra-high molecular weight (UHMW) polyethylene, and ethylene-vinyl acetate (EVA). The illustrated scent block 30 is formed as a single, unitary component. The material used to form the scent block 30 may also be impregnated with a scented oil or similar substance that is designed to slowly release a pleasant scent over a period of time (such as, for example, 30 days). For example, the scent block 30 may be impregnated with a custom blended sent that would be more masculine, i.e., more appealing to males, than is currently available in the market. Also, custom scents may be used as a means of generating advertising or other types of revenue from a customer. Such custom scents may include the launch of a new line of cologne, body spray, or other sense that a customer may wish to have blended into the material used to form the scent block 30.

The hook clip 12 may be formed from a strong, lightweight, and durable plastic, including, but not limited to polypropylene, polyethylene, a polypropylene—EVA blend, and a polyethylene—EVA blend.

In an alternate embodiment, if desired, the material used to form the hook clip 12 may also be impregnated with a scented oil or similar substance that is designed to slowly release a pleasant scent over a period of time (such as, for example, 30 days).

Advantageously, the integrally formed mounting clip 14 allows the scented hook assembly 10 to be placed temporarily, but securely, on a standard restroom stall wall panel having a width between about 0.75 to about 1 inch, such as the stall wall panel 48. As shown in FIG. 3, the hook clip 12 is mounted to the stall wall panel 48 by placing the onto an upper edge of the stall wall panel 48 in the direction of the arrow A, thus inserting the stall wall panel 48 between the planar support wall 16 and the arcuate wall 18.

Additionally, the scented hook assembly 10 is structured and configured to hold items, such as jackets, purses, backpacks, baby bags, and other items carried by a restroom user, securely on the restroom stall wall panel 48 and above the often very dirty restroom floor.

Advantageously, when positioned on the stall wall panel 48, the fins 44 of the scent block 30 release a subtle scent within the restroom stall to freshen the air in an area that often contains unpleasant or otherwise objectionable smells.

Further, the scented hook assembly 10 may be used as an inexpensive replacement for broken, permanently fastened metal hooks, the scented hook assembly 10 may be in addition to unbroken, permanently fastened metal hooks, thus providing additional hooks for a restroom user, while simultaneously freshening the air in and around the restroom stall.

Paid customer scents may be added to the plastic material of the scent block 30 or the hook clip 12 as a means of generating advertising revenue. For example, such paid customer scents may be related to the launch of a new line of cologne, body spray, or any other scent that a customer may wish to have blended into the molded material.

The material used to form the scent block 30 and/or the hook clip 12 may also be formed from, or otherwise include, one or more biodegradable materials to decrease the amount of time needed for the scent block 30 and/or the hook clip 12 to break down in a landfill after use.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A scented hook assembly comprising:
a hook clip; and
a scent block removably attached thereto.

2. The scented hook assembly according to claim 1, wherein the hook clip is configured to be mounted to a support structure and includes:
a body defining a mounting clip; and
a hook;
wherein the mounting clip includes a planar support wall having an inside surface and an outside surface, an arcuate wall having an inside surface and an outside surface, and a closed end wall having an inside surface and an outside surface;
wherein an elongated flex channel is formed at an intersection of the arcuate wall and the closed end wall in at least one of the inside surfaces thereof; and
wherein the hook is formed on the outside surface of the planar support wall and includes a outwardly extending hook portion and a hook base extension that extends outwardly and away from the closed end wall.

3. The scented hook assembly according to claim 2, wherein arcuate indentations are formed on opposing sides of the planar support wall and define finger grips.

4. The scented hook assembly according to claim 3, wherein the flex channels allow the arcuate wall to move outwardly when the hook clip is installed on a support structure.

5. The scented hook assembly according to claim 4, wherein the hook base extension adds structural strength to the hook when a downward force is applied thereto.

6. The scented hook assembly according to claim 5, wherein the hook clip is formed as a single, unitary component from a material that is impregnated with scented oil that is configured to slowly release a predetermined scent over a period of time.

7. The scented hook assembly according to claim 6, wherein the support structure is a restroom stall wall.

8. The scented hook assembly according to claim 7, wherein the scent block is configured to be mounted to the hook clip and includes:
a planar base having an outwardly facing first surface and an inwardly facing second surface;
an elongated bar extending longitudinally parallel to the second surface and defining a mounting slot;
two outwardly extending mounting clips formed on opposing longitudinal ends of the base; and
a plurality of fins extending outwardly from the first surface, each fin having opposing flat faces.

9. The scented hook assembly according to claim 8, wherein the scent block is formed as a single, unitary component from a material that is impregnated with scented oil that is configured to slowly release a predetermined scent over a period of time.

10. The scented hook assembly according to claim 1, wherein the scent block is configured to be mounted to the hook clip and includes:
a planar base having an outwardly facing first surface and an inwardly facing second surface;
an elongated bar extending longitudinally parallel to the second surface and defining a mounting slot;
two outwardly extending mounting clips formed on opposing longitudinal ends of the base; and
a plurality of fins extending outwardly from the first surface, each fin having opposing flat faces.

11. The scented hook assembly according to claim 10, wherein the scent block is formed as a single, unitary component from a material that is impregnated with scented oil that is configured to slowly release a predetermined scent over a period of time.

12. The scented hook assembly according to claim 11, wherein the arcuate wall of the hook clip extends through the mounting slot defined by the elongated bar of the scent block.

13. A hook clip configured to be mounted to a support structure comprising:
a body defining a mounting clip; and
a hook;
wherein the mounting clip includes a planar support wall having an inside surface and an outside surface, an arcuate wall having an inside surface and an outside surface, and a closed end wall having an inside surface and an outside surface;
wherein an elongated flex channel is formed at an intersection of the arcuate wall and the closed end wall in at least one of the inside surfaces thereof; and
wherein the hook is formed on the outside surface of the planar support wall and includes a outwardly extending hook portion and a hook base extension that extends outwardly and away from the closed end wall.

14. The hook clip according to claim 13, wherein arcuate indentations are formed on opposing sides of the planar support wall and define finger grips.

15. The hook clip according to claim 13, wherein the flex channels allow the arcuate wall to move outwardly when the hook clip is installed on a support structure.

16. The hook clip according to claim 13, wherein the hook base extension adds structural strength to the hook when a downward force is applied thereto.

17. The hook clip according to claim 13, wherein the hook clip is formed as a single, unitary component from a material that is impregnated with scented oil that is configured to slowly release a predetermined scent over a period of time.

18. The hook clip according to claim 13, wherein the support structure is a restroom stall wall.

19. A scent block comprising:
a planar base having an outwardly facing first surface and an inwardly facing second surface;
an elongated bar extending longitudinally parallel to the second surface and defining a mounting slot;
two outwardly extending mounting clips formed on opposing longitudinal ends of the base; and
a plurality of fins extending outwardly from the first surface, each fin having opposing flat faces.

20. The scent block according to claim 19, wherein the scent block is formed as a single, unitary component from a material that is impregnated with scented oil that is configured to slowly release a predetermined scent over a period of time.
